(19) United States
(12) Patent Application Publication
(10) Pub. No.: US 2024/0299656 A1
(43) Pub. Date: Sep. 12, 2024

(54) Infusion Device, Control Method, and Computer-Readable Storage Medium

(71) Applicant: Medcaptain Medical Technology Co., Ltd., Shenzhen (CN)

(72) Inventors: Shengping LI, Shenzhen (CN); Ping LI, Shenzhen (CN); Longyu WU, Shenzhen (CN); Yazhou TANG, Shenzhen (CN)

(73) Assignee: Medcaptain Medical Technology Co., Ltd., Shenzhen (CN)

(21) Appl. No.: 18/595,830

(22) Filed: Mar. 5, 2024

(30) Foreign Application Priority Data
Mar. 10, 2023 (CN) 202310072905.9

(51) Int. Cl. A61M 5/172; G16H 20/17; G16H 40/67

(52) U.S. Cl. CPC A61M 5/1723; G16H 20/17; G16H 40/67; A61M 2205/3303

(57) ABSTRACT

The invention provides an infusion device, a control method and a computer-readable storage medium. A target object, such as a patient, is infused according to a set infusion parameter and a current first physiological parameter of the target object is determined. A target infusion parameter is then determined according to the target parameter when the first physiological parameter falls outside a preset range. The target object is infused according to the target infusion parameter. The target parameter includes the first physiological parameter, a weight of the target object and/or a constant infusion parameter. When the physiological parameters of the target object fall outside the preset range, the infusion device automatically adjusts the infusion parameters of the infusion device, without requiring the healthcare personnel to manually switch the operating mode of the infusion device for adjustment of the infusion parameters, thereby improving the efficiency of the infusion device.

### REFERENCE TO RELATED APPLICATIONS

This application claims priority of Chinese Patent Application No. 202310072905.9, entitled “Infusion Device, Control Method, and Computer-Readable Storage Medium” filed with the China National Intellectual Property Administration on Mar. 10, 2023, which is incorporated herein by reference in its entirety.

### TECHNICAL FIELD

The invention relates to the field of medical devices, and more particularly, to an infusion device, control method, and computer-readable storage medium.

### BACKGROUND ART

With the development of the medical industry, medical devices are becoming more and more intelligent, for example, infusion device can automatically infuse patients.

In the process of drug infusion, healthcare personnel set the infusion parameters of the infusion device according to their own experience, so that the infusion device will infuse the patient according to the set infusion parameters. When the patient's physiological parameters reach the expectations of the healthcare personnel, the healthcare personnel will then switch the infusion device to other modes, so as to enable the infusion device to set the infusion parameters in line with the current state of the patient.

As can be seen, mode switching of infusion parameters in the infusion device takes place manually, and such manual operation takes a long time; this results in low operational efficiency of the infusion device.

### SUMMARY OF THE INVENTION

The invention provides an infusion device, a control method and a computer-readable storage medium for solving the problem of low operating efficiency of an infusion device.

On the one hand, the invention provides a method for controlling an infusion device, comprising:
infusing a target object according to a set infusion parameter, and acquiring a current first physiological parameter of the target object;
determining a target infusion parameter according to a target parameter when the first physiological parameter falls outside of a preset range of physiological parameters, and infusing the target object according to the target infusion parameter, wherein the target parameter comprises the first physiological parameter, a weight of the target object, or a constant infusion parameter.

In an embodiment, the step of infusing the target object according to the set infusion parameter comprises:
infusing the target object according to the set infusion parameter when the infusion device enters a first operating mode;
the step of determining the target infusion parameter according to the target parameter comprises:
determining a second operating mode to be switched from the first operating mode, and running the second operating mode;
determining the target parameter according to the second operating mode, and determining the target infusion parameter according to the target parameter.

In an embodiment, the step of determining the target infusion parameter according to a target parameter comprises:
acquiring a physiological characteristic of the target object;
obtaining a target model matched to the physiological characteristics;
inputting the target parameters into the target model, and obtaining the target infusion parameters output from the target model.

In an embodiment, the step of infusing the target object according to the target infusion parameters comprises:
acquiring a second physiological parameter of the target object;
infusing the target object according to the set infusion parameter when the second physiological parameter is in a preset range of the physiological parameters.

In an embodiment, before the step of infusing the target object according to the set infusion parameter, the method further comprises:
outputting a control page;
when detecting a completion operation of an infusion parameter triggered on the control page, obtaining an input infusion parameter according to the completion operation, and determining the set infusion parameter according to the input infusion parameter.

In an embodiment, the method further comprises:
outputting a setting page of a range of physiological parameters;
when detecting a setting operation of the range of physiological parameters in the setting page, obtaining an input physiological parameter according to the setting operation, and setting a preset range of physiological parameter according to the input physiological parameter.

In an embodiment, the step of acquiring the current first physiological parameter of the target object comprises:
sending an acquisition request of a physiological parameter to an acquisition device of the physiological parameter, wherein the acquisition device is configured to acquire the physiological parameter of the target object;
receiving the current first physiological parameter of the target object sent by the acquisition device.

In an embodiment, the step of acquiring the current first physiological parameter of the target object comprises:
acquiring a physiological characteristic of the target object;
inputting the physiological characteristic and the set infusion parameters into a prediction model to obtain the current first physiological parameter of the target object output by the prediction model.

Additionally, the invention further provides an infusion device comprising:
an infusion module configured to infuse a target object according to a set infusion parameter, and acquire a current first physiological parameter of the target object;
a determination module configured to determine a target infusion parameter according to a target parameter when the first physiological parameter falls outside of a preset range of physiological parameters, and infuse the target object according to the target infusion parameter, wherein the target parameter comprises the first physiological parameter, a weight of the target object, or a constant infusion parameter.

The invention further provides an infusion device comprising: a memory and a processor;
the memory is configured to store a computer-executable instruction; and
the processor is configured to execute the computer-executable instructions stored in the memory, such that the processor performs a control method of an infusion device, comprising:
infusing a target object according to a set infusion parameter, and acquiring a current first physiological parameter of the target object;
determining a target infusion parameter according to a target parameter when the first physiological parameter falls outside of a preset range of physiological parameters, and infusing the target object according to the target infusion parameter, wherein the target parameter comprises the first physiological parameter, a weight of the target object, or a constant infusion parameter.

The invention further provides a computer-readable storage medium, comprising a computer-executable instruction, wherein the computer instructions is executed by the processor to implement a control method of an infusion device, comprising:
infusing a target object according to a set infusion parameter, and acquiring a current first physiological parameter of the target object;
determining a target infusion parameter according to a target parameter when the first physiological parameter falls outside of a preset range of physiological parameters, and infusing the target object according to the target infusion parameter,
wherein the target parameter comprises the first physiological parameter, a weight of the target object, or a constant infusion parameter.

In the infusion device, using the control method and the computer-readable storage medium provided in the invention, the infusion device can automatically adjust the infusion parameters of the infusion device by means of the physiological parameters, the body weight of the target object, or the constant infusion parameters when detecting that the physiological parameters of the target object have exceeded the preset range of the physiological parameters. It does not require healthcare personnel to manually switch the operating mode of the infusion device to adjust the infusion parameters and this improves the operation efficiency of the infusion device.

### BRIEF DESCRIPTION OF THE DRAWINGS

The accompanying drawings herein, which are incorporated into and form a part of the specification, illustrate embodiments consistent with the invention and are used in conjunction with the specification to explain the principles of the invention.

FIG. 1 shows a schematic diagram of an application scenario of a control method of an infusion device according to one embodiment of the invention.

FIG. 2 shows a schematic flowchart of the control method of the infusion device according to a first embodiment of the invention.

FIG. 3 shows a schematic flowchart of the control method of the infusion device according to a second embodiment of the invention.

FIG. 4 shows a schematic flowchart of the control method of the infusion device according to a third embodiment of the invention.

FIG. 5 shows a schematic flowchart of the control method of the infusion device according to a fourth embodiment of the invention.

FIG. 6 shows a schematic flowchart of the control method of the infusion device according to a fifth embodiment of the invention.

FIG. 7 shows a schematic diagram of a module of the infusion device according to another embodiment of the invention.

FIG. 8 shows a schematic diagram of a hardware structure of the infusion device according to an embodiment of the invention.

By means of the above accompanying drawings, definite embodiments of the invention are shown and will be described in more detail later. These accompanying drawings and textual descriptions are not intended to limit the scope of the presently conceived disclosure in any way, but rather to illustrate the concepts of the invention for those skilled in the art by reference to particular embodiments.

### DETAILED DESCRIPTION

Example of embodiments will be described herein in detail, which are represented in the accompanying drawings. When the following description relates to the accompanying drawings, the same numerals in the different accompanying drawings indicate the same or similar elements unless otherwise indicated. The embodiments described in the following example embodiments do not represent all embodiments consistent with the invention. Rather, they are only examples of devices and methods consistent with some aspects of the invention as detailed in the appended claims.

As the medical industry evolves, medical devices are becoming more intelligent. For example, infusion devices can be automated to administer an infusion to a patient.

The inventors of this invention have found that, during the process of drug infusion, healthcare personnel set infusion parameters of the infusion device according to their own experience, thereby causing the infusion device to infuse the patient according to the set infusion parameter. When the physiological parameters of the patient reach the expectations of the healthcare personnel, the healthcare personnel will then switch the infusion device to other modes, so as to facilitate the infusion device to set the infusion parameters in line with the current state of the patient. This involves a manual operation for the mode switching of the infusion parameters in the infusion device, and this manual operation takes a long time, resulting in a low operation efficiency of the infusion device.

The inventors therefore thought that the infusion device, when detecting that the physiological parameters of the target object exceed a preset range of physiological parameters, can automatically adjust the infusion parameters of the infusion device by means of the physiological parameters, the body weight of the target object, or a constant infusion parameter, so that there is no need for healthcare personnel to manually switch the operating mode of the infusion device for adjusting the infusion parameters, thereby improving the operation efficiency of the infusion device.

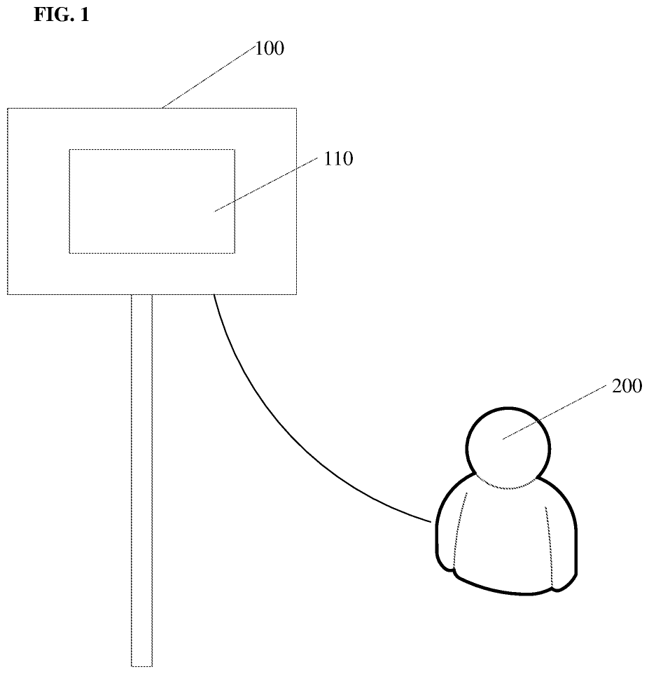

The invention provides a control method of an infusion device. As shown in FIG. 1, the infusion device 100 infuses a target object 200 through an infusion tube. Infusion refers to inputting a drug or a nutrient solution to the target object. The infusion device 100 is provided with a control panel 110, and the infusion parameters may be set via the control panel 110, in which the set infusion parameter is defined as a set infusion parameter. The infusion device 100 may first infuse the target object 200 according to the set infusion parameter, and obtain physiological parameters of the target object 200, which may be obtained by an acquisition device worn by the target object 200, or may be predicted by the infusion device 100 for the target object. If the physiological parameters are out of the preset range of the physiological parameters, the infusion device 100 automatically adjusts the infusion parameters of the target object 200 by means of the physiological parameters, the weight of the target object, or the constant infusion parameters of the infusion device 100.

The technical solution of the invention and how the technical solution of the invention solves the above-described technical problems are described in detail in the following description of specific embodiments. These following specific embodiments may be combined with each other, and the same or similar concepts or processes may not be repeated in certain embodiments. Embodiments of the invention will be described below in conjunction with the accompanying drawings.

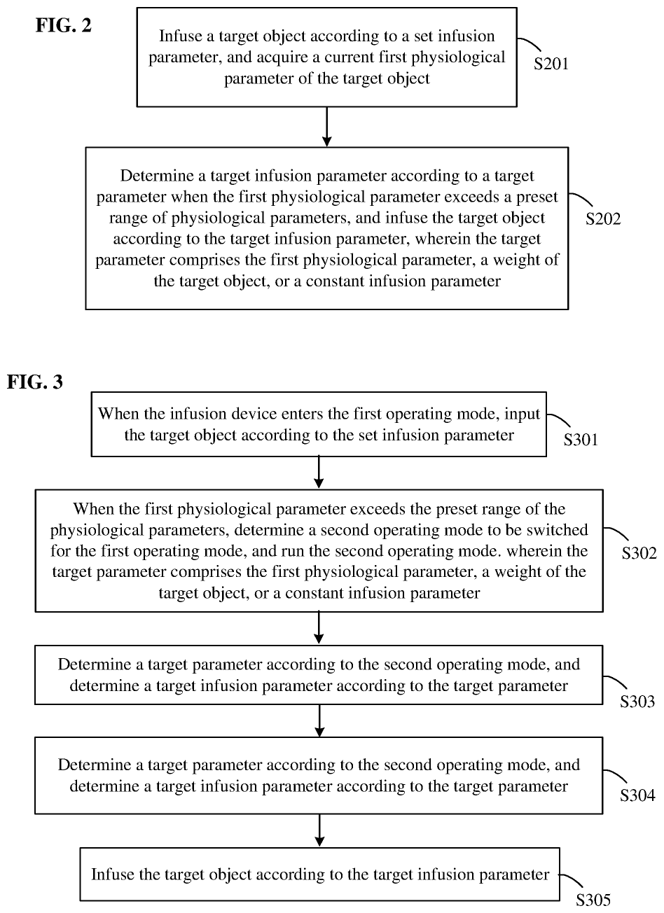

As shown in FIG. 2, FIG. 2 shows a control method for an infusion device according to the first embodiment of the invention, and the control method of the infusion device includes the following steps.

Step S201, the target object is infused according to the set infusion parameter, and a current first physiological parameter of the target object is acquired.

In this embodiment, the execution subject is an infusion device, and the infusion device may be a device that performs infusion for the human body. For example, the infusion device may be an infusion pump.

When the infusion device performs the infusion on the target object, it first infuses the target object according to the set infusion parameter. The set infusion parameter may be entered by healthcare personnel on the infusion device in advance. The target object refers to an object to be infused by the infusion device, and the target object may be a patient.

In an embodiment, the infusion device is provided with a display screen, and the infusion device outputs a displayed control page on which the healthcare personnel may enter the infusion parameters. The infusion device, when detecting a completion operation of the infusion parameters triggered on the control page, obtains the input infusion parameters according to the completion operation and determines the set infusion parameters according to the input infusion parameters. The completion operation may be a click operation of a confirmation button on the control page, i.e., the healthcare practitioner clicks the confirmation button after inputting the infusion parameters on the control page, and the infusion device detects the completion operation.

In another embodiment, the infusion device may be communicatively connected to the control device, and the healthcare personnel may input the infusion parameters to the infusion device via the control device, i.e., the control device transmits the infusion parameters input by the healthcare personnel as well as an identification of the healthcare personnel to the infusion device, and the infusion device determines, according to the identification, whether or not the healthcare personnel has the setting privilege. If it is determined that the healthcare personnel has the setting privilege, the infusion device then takes the infusion parameters as the set infusion parameters. The control device may be a device loaded with a control program for the infusion device. For example, the control device may be a portable terminal of the healthcare personnel; or, the control device may be a computer.

When infusing the target object according to the set infusion parameter, the infusion device also obtains a current physiological parameter of the target object in real time or at regular intervals, and the current physiological parameter is defined as a first physiological parameter. The first physiological parameter is related to the drug infused by the infusion device. For example, the drug infused by the infusion device is an anesthetic, then the first physiological parameter is a Bispectral index (BIS, brain electric dual frequency index) of the target object. The set infusion parameters includes the set infusion concentration, the set infusion volume, and the set infusion time. The infusion device is communicatively connected to the acquisition device for the physiological parameters, and the infusion device sends an acquisition request for the physiological parameters to the acquisition device when it needs to acquire the physiological parameters, and the acquisition device, upon receiving the acquisition request, sends the current first physiological parameter of the target object to the infusion device, i.e., the infusion device receives the current first physiological parameter of the target object sent by the acquisition device.

Step S202, when the first physiological parameter falls outside of a preset range of physiological parameters, a target infusion parameter is determined according to the target parameter, and the target object is infused according to the target infusion parameter. The target parameter includes the first physiological parameter, the weight of the target object, or the constant infusion parameter.

After acquiring the first physiological parameter, the infusion device determines whether the first physiological parameter exceeds the preset range of physiological parameters. For example, when the drug infused by the infusion device is an anesthetic, the preset range of physiological parameters refers to a BIS range. The preset range of physiological parameters may be set by the healthcare personnel. Furthermore, the physiological parameters included in the preset range of physiological parameters are not limited to BIS, but may also be other cardiac parameters.

In an embodiment, the infusion device is provided with a display screen, and the infusion device outputs a setting page for the physiological parameter range. The healthcare personnel may enter the physiological parameter range on the setting page. The infusion device, when detecting a setting operation of the physiological parameter range triggered on the setting page, acquires the input physiological parameter according to the setting operation and sets a preset range of physiological parameters according to the input physiological parameter. The setting operation may be a click operation of a setting button on the control page, i.e., after the healthcare personnel inputs the physiological parameters on the setting page, the healthcare personnel clicks the setting button, and the infusion device detects the setting operation. It should be noted that the healthcare personnel needs to input two physiological parameters, so that the infusion device can generate the preset range of physiological parameters according to the two physiological parameters.

In another embodiment, the infusion device may be communicatively connected to the control device, and the healthcare personnel may input the physiological parameters to the infusion device via the control device. That is, the control device transmits the physiological parameters inputted by the healthcare personnel as well as an identification of the healthcare personnel to the infusion device, and the infusion device determines, according to the identification, whether or not the healthcare personnel has the setting privilege. If it is determined that the healthcare personnel has the setting privilege, the infusion device sets the preset range of physiological parameters according to the physiological parameters.

In this embodiment, the set infusion parameters is set according to the experience of the healthcare personnel, and the preset range of physiological parameters is also set according to the experience of the healthcare personnel. When the first physiological parameter exceeds the preset range of the physiological parameters, it can be determined that the state of the target object is stable, and there is no need to perform infusion according to the set infusion parameter, i.e., there is no need for the healthcare personnel to manually control the infusion parameters of the infusion device, so the infusion device can determine the infusion parameters on its own and perform the infusion of the target object according to the set infusion parameter. When the infusion device determines that the first physiological parameter exceeds that preset range of physiological parameters, it determines the target infusion parameter according to the target parameters, thereby controlling the infusion device to infuse the target object according to the target infusion parameters. The target parameter includes the first physiological parameter, the weight of the target object, or a constant infusion parameter.

In this embodiment, when the infusion device detects that the physiological parameter of the target object falls outside of a preset range of physiological parameters, it automatically adjusts the infusion parameters of the infusion device by the physiological parameter, the body weight of the target object, or the constant infusion parameter, without requiring the healthcare personnel to manually switch the operating mode of the infusion device for adjusting the infusion parameters, which improves the operation efficiency of the infusion device.

As shown in FIG. 3, FIG. 3 shows a control method of the infusion device according to the second embodiment of the invention. According to the first embodiment, step S201 includes step S301, and step S202 includes step S302, step S303, and step S304.

Step S301, when the infusion device enters the first operating mode, the target object is infused according to the set infusion parameter.

In this embodiment, the infusion device first operates according to the first operating mode, and the first operating mode is, for example, a total intravenous controlled anesthesia (TIVA) mode, in which the infusion device infuses the target object according to the infusion parameters set by the healthcare personnel. That is, when the infusion device enters the first operating mode, the infusion device infuses the target object according to the set infusion parameter.

Step S302, when the first physiological parameter exceeds the preset range of the physiological parameters, a second operating mode to be switched for the first operating mode is determined, and the infusion device is run in the second operating mode.

Step S303, a target parameter is determined according to the second operating mode, and a target infusion parameter is determined according to the target parameter.

Step S304, the target object is infused according to the target infusion parameter, in which the target parameter includes the first physiological parameters, the weight of the target object, or the constant infusion parameters.

When the first physiological parameter exceeds the preset range of the physiological parameters, the infusion device determines a second operating mode in which the first operating mode is to be switched, thereby determining the target parameter according to the second operating mode, then determining the target infusion parameter according to the target parameter, and finally infusing the target object with the infusion parameter according to the target infusion parameter. The second operating modes are different, and the target parameters are different. There is a plurality of operating modes in the infusion device, and the currently running operating mode is defined as the first operating mode. For example, the second operating mode to be switched may be determined by at least one of the information such as the drug infused in the target object, the weight parameter of the target object, the physiological parameter, and the like. Each of the operating modes is associated with a drug, a weight parameter, and a physiological parameter. The infusion device acquires the infused drug, the weight parameter of the target object, and the physiological parameter of the target object, and then compares and matches the selected infused drug, the weight parameter of the target object, and the physiological parameter with the drug, the weight parameter, and the physiological parameter associated with each of the operating modes, so as to determine the second operating mode to be determined. For example, when the drug currently being infused is an anesthetic, the operating modes associated with the anesthetic include a weight mode, an auto-adjustment mode, and a constant rate mode. If it is determined that the target object is a thin child according to the weight parameter of the target object, and only the weight mode is associated with the infusion with child and weight, and thus the weight mode is the second operating mode to be switched. If it is determined that the target object is an adult according to the physiological parameter of the target object, since adult infusion is associated with an auto-adjustment mode as well as a constant rate mode, and the infusion parameters are determined to require automatic adjustment for the infusion of the drug through the physiological parameters of the target object, the auto-adjustment mode serves as the second operating mode to be switched. It is to be noted that the above is merely for illustrative purposes and does not limit the manner in which the second operating mode is determined.

In this embodiment, the operating mode of the infusion device is adjusted to fit the infusion situation of the target object through the drug to be infused and the physiological characteristics of the target object, resulting in a high infusion flexibility of the infusion device.

In an embodiment, when the second operating mode is an auto-adjustment mode, then the first physiological parameter is determined as the target parameter. For example, the auto-adjustment mode may be a target-controlled infusion (TCI) mode, then the first physiological parameter is a BIS value.

In another embodiment, when the second operating mode is the weight mode, the weight of the target subject is determined as the target parameter, i.e., the weight of the target subject should be referred to when determining the target infusion parameter. For example, a correction coefficient may be determined by the weight of the target object, and a target correction parameter is obtained by correcting the first infusion parameter with the correction coefficient. The first infusion parameter is an infusion parameter set in the weight mode.

In yet another example, when the second operating mode is the constant rate mode, the constant infusion parameter is determined as the target parameter, i.e., the infusion device infuses the target object according to the constant infusion parameter. The constant infusion parameter include a constant flow rate, a constant infusion time, a set infusion concentration, and a constant infusion volume.

In the conventional technologies, the mode of the infusion device needs to be switched manually, e.g., the infusion device outputs a voice prompt when it detects that the mode switching needs to be performed in order to wait for the manual switching. In contrast, in this embodiment, the infusion device is capable of automatically switching the first operating mode to the second operating mode. Further, the mode switching of the infusion device includes automatic switching as well as manual switching, and the automatic switching as well as the manual switching of the infusion device needs to be set. If a plurality of infusion devices are connected to the same local area network, in which one of the working infusion devices carries out the automatic switching of modes, and the other infusion devices also carry out the automatic switching of modes when they are working, so as to avoid setting up the automatic switching of modes for each of the infusion devices one by one.

In this embodiment, after the infusion device is switched from the first operating mode to the second operating mode, the target parameter is determined by the second operating mode, so that the target infusion parameter matching the second operating mode is determined according to the target parameter.

As shown in FIG. 4, FIG. 4 shows a control method of the infusion device according to the third embodiment of the invention. According to the second embodiment, step S202 further includes the following steps.

Step S401, a physiological characteristic of the target object is acquired.

Step S402, a target model matched with the physiological characteristic is acquired.

Step S403, a target parameter is inputted to the target model, and a target infusion parameter output by the target model is obtained.

In this embodiment, the infusion device may find a suitable operating mode to set the target infusion parameters for the infusion device through the difference in the physiological characteristics of the target object.

The operating mode is obtained by using, as ML/AI training data, the data among the user, the target parameters, and the infusion parameters of the user with not much difference in physiological characteristics. The physiological characteristics include weight, age, gender, height, and the like of the users, i.e., the physiological characteristics, the target parameters, and the infusion parameters of the users belonging to the same group used in the infusion are used for training to obtain the operating mode corresponding to the group. The operating mode is stored in association with the identity of the group.

The infusion device stores information about the target object, and the infusion device obtains physiological characteristics of the target object from the information about the target object. The infusion device can determine the group to which the target object belongs by the physiological characteristics, so as to obtain an operating mode matched with the target object as the target model through the group.

The infusion device inputs the target parameters into the target model, and the target model outputs the target infusion parameter according to the target parameter.

In addition, the operating mode associated with the identification of the group is stored in a server, and the infusion device sends the physiological characteristics of the target object to the server to request the server to send the target model matched with the target object, and then inputs the target parameters into the target model to obtain the target infusion parameters.

In this embodiment, the infusion device acquires the physiological characteristics of the target object, thereby obtaining a target model matched with the physiological characteristics. Then the target parameters are inputted into the target model to obtain the target infusion parameters, i.e., the infusion device accurately determines the target infusion parameters according to the physiological characteristics of the target object.

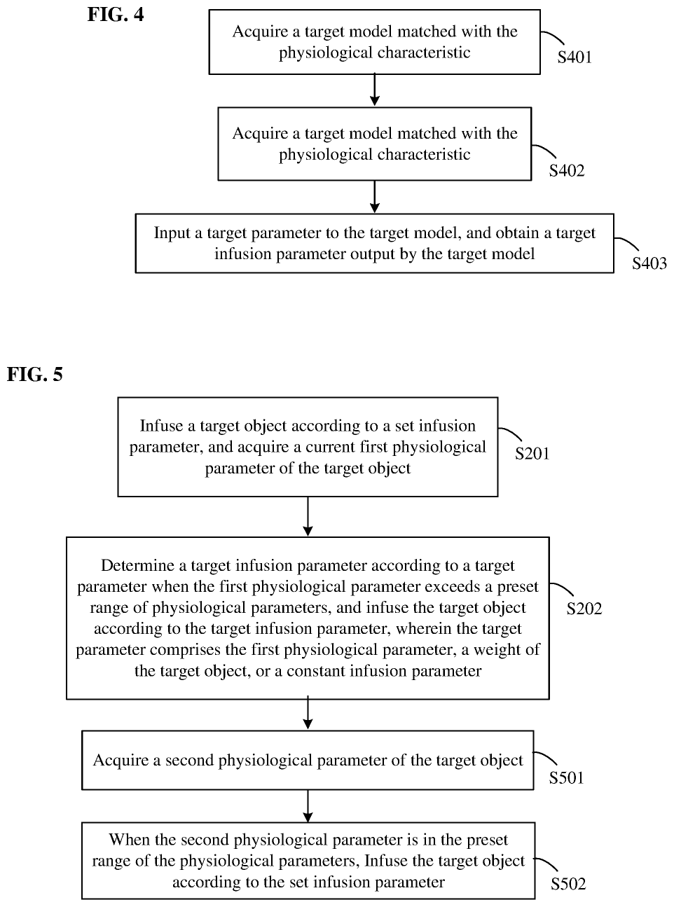

As shown in FIG. 5, FIG. 5 shows a control method of the infusion device according to the fourth embodiment of the invention. According to any one of the first to third embodiments, after step S202, the method further includes the following steps.

Step S501, a second physiological parameter of the target object is acquired.

Step S502, when the second physiological parameter is in the preset range of the physiological parameters, the target object is infused according to the set infusion parameter.

In this embodiment, after infusing the target object according to the target infusion parameters, the infusion device continues to acquire physiological parameters of the target object, and the acquired physiological parameters are defined as the second physiological parameters.

The infusion device determines whether the second physiological parameter is in a preset range of physiological parameters. If the second physiological parameter is in the preset range of the physiological parameters, the infusion device is required to perform the infusion of the target object according to the set infusion parameter.

When the infusion device is in the first operating mode, the target object is infused according to the set infusion parameter. If the physiological parameter of the target object exceeds the preset range of the physiological parameters, the infusion device is switched to the second operating mode, so that the infusion device infuses the target object according to the target parameters determined by the target parameters. If the physiological parameter of the target object is in the preset range of the physiological parameters, the infusion device is switched to the first operating mode to operate according to the set infusion parameter.

In this embodiment, the infusion device acquires the second physiological parameter of the target object, and if the second physiological parameter is in the preset range of the physiological parameters, the infusion device infuses the target according to the set infusion parameter.

As shown in FIG. 6, FIG. 6 shows a control method of the infusion device according to a fifth embodiment of the invention. According to any one of the first to fourth embodiments, step S201 further includes the following steps.

Step S601, a physiological characteristic of the target object is acquired.

Step S602, the physiological characteristic and the set infusion parameter are inputted to a prediction model to obtain the current first physiological parameter of the target object output by the prediction model.

In this embodiment, the infusion device is provided with the prediction model, and the prediction model may be used to predict the first physiological parameter of the user. For example, the first physiological parameter may be a concentration of an anesthetic agent in the user's body, i.e., the prediction model may predict the concentration of the anesthetic agent in the user's body. The prediction model is trained by the physiological characteristics of the user, the infusion parameters of the infusion device employed by the user, and the physiological parameters of the user.

The device obtains the physiological characteristic of the target object, and the physiological characteristic include weight, height, gender, and age of the target object. The device inputs the set infusion parameters and the physiological characteristics into the prediction model to obtain a first current physiological parameter of the target object output by the prediction model.

In this embodiment, the infusion device can predict the current first physiological parameter of the target object by inputting the physiological parameters of the target object and the set infusion parameters into the prediction model, and there is no need to use the device to collect the physiological parameters of the target object, which saves costs.

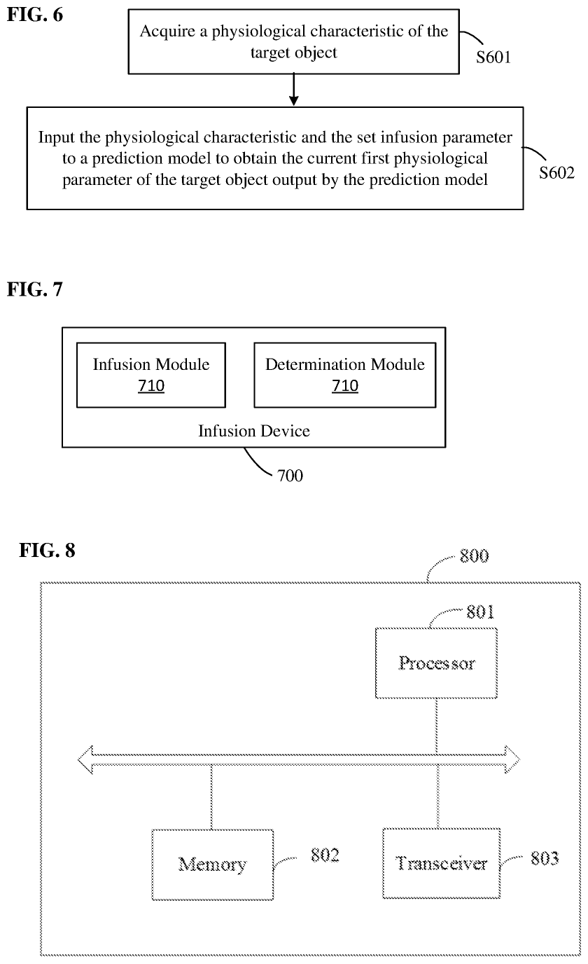

The invention further provides an infusion device. As shown in FIG. 7, the infusion device 700 includes the infusion module 710 and the determination module 720.

The infusion module 710 is configured to infuse the target object according to set infusion parameter, and obtain a current first physiological parameter of the target object.

The determination module 720 is configured to determine a target infusion parameter according to a target parameter when the first physiological parameter exceeds the preset range of physiological parameters, and infuse the target object according to the target infusion parameter. The target parameter includes the first physiological parameter, the weight of the target object, or a constant infusion parameter.

In an embodiment, the infusion device 700 includes:

An infusion module 710 configured to infuse the target object according to the set infusion parameter when the infusion device enters the first operating mode; and
a determining module 720 configured to determine a second operating mode to be switched by the first operating mode, and run the second operating mode;

The determining module 720 is further configured to determine a target parameter according to the second operating mode and determine a target infusion parameter according to the target parameter.

In an embodiment, the infusion device 700 includes:
a first acquisition module configured to acquire a physiological characteristic of the target subject;
a second acquisition module configured to acquire a target model matched to the physiological characteristic; and
an input module configured to input the target parameter to the target model to obtain target infusion parameter output by the target model.

In an embodiment, the infusion device 700 includes:
an acquisition module configured to acquire a second physiological parameter of the target subject;

An infusion module 710 configured to infuse the target object according to the set infusion parameter when the second physiological parameter is in the preset range of the physiological parameter.

In an embodiment, the infusion device 700 includes:
an output module configured to output a control page;
an acquisition module configured to acquire an input infusion parameter according to a completion operation of the infusion parameter triggered on the control page when detecting the completion operation, and determine the set infusion parameter according to the input infusion parameter.

In an embodiment, the infusion device 700 includes:
an output module configured to output a setting page for a range of physiological parameters;
an acquisition module configured to acquire input physiological parameters according to the setting operation on the setting page for the range of physiological parameters when detecting the setting operation in the range of physiological parameters triggered on the setting page, and determine the preset range of physiological parameters according to the input physiological parameters.

In an embodiment, the infusion device 700 includes:
a sending module configured to send an acquisition request of the physiological parameters to an acquisition device of the physiological parameters, in which the acquisition device is configured to acquire the physiological parameters of the target object;
a receiving module configured to receive a current first physiological parameter of the target object sent by the acquisition device.

In an embodiment, the infusion device 700 includes:
an acquisition module configured to acquire a physiological characteristic of the target object;
an input module configured to input the physiological characteristics as well as the set infusion parameters to a prediction model to obtain the current first physiological parameter of the target object output by the prediction model.

FIG. 8 is a diagram of a hardware structure of an infusion device illustrated according to an embodiment.

The infusion device 800 may include: a processor 801, such as a CPU, a memory 802, a transceiver 803. It will be appreciated by those skilled in the art that the structure illustrated in FIG. 8 does not constitute a limitation of the infusion device, and may include more or fewer components than illustrated, or a combination of certain components, or a different arrangement of components. The memory 802 may be implemented by any type of volatile or non-volatile storage device or a combination thereof, such as static random access memory (SRAM), electrically erasable programmable read-only memory (EEPROM), erasable programmable read-only memory (EPROM), programmable read-only memory (PROM), read-only memory (ROM), magnetic memory, flash memory, a disk or optical disk.

The processor 801 may invoke a computer program stored within the memory 802 to accomplish all or some of the steps of the control method of the infusion device described above.

The transceiver 803 is configured to receive information sent by the external device and to send information to the external device.

Anon-transitory computer-readable storage medium that, when computer instructions in the storage medium are executed by a processor of an infusion device, enables the infusion device to perform the above-described control method of the infusion device.

A computer program product including a computer program that, when the computer program is executed by a processor of an infusion device, enables the infusion device to perform the control method of the infusion device.

Other embodiments of the invention will readily come to mind to those skilled in the art upon consideration of the specification and practice of the invention disclosed herein. The invention is intended to cover any variations, uses, or adaptations of the invention that follow the general principles of the invention and include common knowledge or customary technical means in the art not disclosed herein. The specification and embodiments are to be regarded as exemplary only, and the true scope and spirit of the invention is indicated by the following claims.

It should be understood that the invention is not limited to the precise structure which has been described above and illustrated in the accompanying drawings, and that various modifications and alterations may be made without departing from its scope. The scope of the invention is limited only by the appended claims.

1. A method for controlling an infusion device, comprising:
infusing a target object according to a set infusion parameter, and acquiring a current first physiological parameter of the target object; and
determining a target infusion parameter according to a target parameter when the first physiological parameter falls outside of a preset range of physiological parameters, and automatically switching the infusion device to infuse the target object according to the target infusion parameter, wherein the target parameter comprises at least one of: the first physiological parameter, a weight of the target object, and a constant infusion parameter.

2. The method of claim 1, wherein the step of infusing the target object according to the set infusion parameter comprises:
infusing the target object according to the set infusion parameter when the infusion device enters a first operating mode;
the step of determining the target infusion parameter according to the target parameter comprises:
determining a second operating mode to be switched by the first operating mode, and running the second operating mode; and
determining the target parameter according to the second operating mode, and determining the target infusion parameter according to the target parameter.

3. The method of claim 1, wherein the step of determining the target infusion parameter according to the target parameter comprises:
acquiring a physiological characteristic of the target object;
obtaining a target model matched to the physiological characteristics; and
inputting the target parameters into the target model, and obtaining the target infusion parameters output from the target model.

4. The method of claim 1, wherein after the step of infusing the target object according to the target infusion parameters, the method further comprises:
acquiring a second physiological parameter of the target object; and
infusing the target object according to the set infusion parameter when the second physiological parameter is in the preset range of the physiological parameters.

5. The method of claim 1, wherein before the step of infusing the target object according to the set infusion parameter, the method further comprises:
outputting a control page; and
when detecting a completion operation of an infusion parameter triggered on the control page, obtaining an input infusion parameter according to the completion operation, and determining the set infusion parameter according to the input infusion parameter.

6. The method of claim 1, further comprising:
outputting a setting page of a range of physiological parameters; and
when detecting a setting operation of the range of physiological parameters in the setting page, obtaining an input physiological parameter according to the setting operation, and setting a preset range of physiological parameter according to the input physiological parameter.

7. The method of claim 1, wherein the step of acquiring the current first physiological parameter of the target object comprises:
sending an acquisition request of a physiological parameter to an acquisition device of the physiological parameter, wherein the acquisition device is configured to acquire the physiological parameter of the target object; and
receiving the current first physiological parameter of the target object sent by the acquisition device.

8. The method of claim 1, wherein the step of acquiring the current first physiological parameter of the target object comprises:
acquiring a physiological characteristic of the target object; and
inputting the physiological characteristic and the set infusion parameters into a prediction model to obtain the current first physiological parameter of the target object output by the prediction model.

9. An infusion device, comprising:
an infusion module configured to infuse a target object according to a set infusion parameter, and acquire a current first physiological parameter of the target object; and
a determination module configured to determine a target infusion parameter according to a target parameter when the first physiological parameter falls outside of a preset range of physiological parameters, and automatically switching the infusion device to infuse the target object according to the target infusion parameter, wherein the target parameter comprises at least one of: the first physiological parameter, a weight of the target object, and a constant infusion parameter.

10. An infusion device comprising:
a memory and a processor;
said memory being configured to store a computer-executable instruction; and
said processor being configured to execute the computer-executable instructions stored in the memory, such that the processor is caused to control the infusion device by:
infusing a target object according to a set infusion parameter, and acquiring a current first physiological parameter of the target object; and
determining a target infusion parameter according to a target parameter when the first physiological parameter falls outside of a preset range of physiological parameters, and automatically switching the infusion device to infusing the target object according to the target infusion parameter, wherein the target parameter comprises at least one of the first physiological parameter, a weight of the target object, and a constant infusion parameter.

11. The infusion device of claim 10, wherein the processor is configured to infuse the target object according to the set infusion parameter by:
infusing the target object according to the set infusion parameter when the infusion device enters a first operating mode;
and the processor is configured to determine the target infusion parameter according to the target parameter by:
determining a second operating mode to be switched by the first operating mode, and running the second operating mode; and
determining the target parameter according to the second operating mode, and determining the target infusion parameter according to the target parameter.

12. The infusion device of claim 10, wherein the processor is configured to determine the target infusion parameter according to the target parameter by:
acquiring a physiological characteristic of the target object;
obtaining a target model matched to the physiological characteristics; and
inputting the target parameters into the target model, and obtaining the target infusion parameters output from the target model.

13. The infusion device of claim 10, wherein after the step of infusing the target object according to the target infusion parameters, the processor is further configured to:
acquire a second physiological parameter of the target object; and
infuse the target object according to the set infusion parameter when the second physiological parameter is in the preset range of the physiological parameters.

14. The infusion device of claim 10, wherein before the step of infusing the target object according to the set infusion parameter, the processor is further configured to:
output a control page; and
when detecting a completion operation of an infusion parameter triggered on the control page, obtain an input infusion parameter according to the completion operation, and determining the set infusion parameter according to the input infusion parameter.

15. The infusion device of claim 10, wherein the processor is further configured to:
output a setting page of a range of physiological parameters;
when detecting a setting operation of the range of physiological parameters in the setting page, obtain an input physiological parameter according to the setting operation, and set a preset range of physiological parameter according to the input physiological parameter.

16. The infusion device of claim 10, wherein the processor acquire the current first physiological parameter of the target object by:
sending an acquisition request of a physiological parameter to an acquisition device of the physiological parameter, wherein the acquisition device is configured to acquire the physiological parameter of the target object;
receiving the current first physiological parameter of the target object sent by the acquisition device.

17. The infusion device of claim 10, wherein the processor acquires the current first physiological parameter of the target object by:
acquiring a physiological characteristic of the target object;
inputting the physiological characteristic and the set infusion parameters into a prediction model to obtain the current first physiological parameter of the target object output by the prediction model.
